(19) United States
(12) Patent Application Publication
(10) Pub. No.: US 2008/0248514 A1
(43) Pub. Date: Oct. 9, 2008

(54) ELECTROCHEMICAL METHOD FOR GLUCOSE QUANTIFICATION, GLUCOSE DEHYDROGENASE COMPOSITION, AND ELECTROCHEMICAL SENSOR FOR GLUCOSE MEASUREMENT

(75) Inventors: Kazuki INAMORI, Tsuruga-shi (JP); Masao KITABAYASHI, Tsuruga-shi (JP); Yuji TSUJI, Tsuruga-shi (JP); Yoshiaki NISHIYA, Tsuruga-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: TOYO BOSEKI KABUSHIKI KAISHA, Osaka-shi (JP)

(21) Appl. No.: 12/031,834

(22) Filed: Feb. 15, 2008

Related U.S. Application Data
(60) Provisional application No. 60/908,852, filed on Mar. 29, 2007.

(30) Foreign Application Priority Data
Feb. 20, 2007 (JP) 2007-039133
Mar. 22, 2007 (JP) 2007-075022
Mar. 22, 2007 (JP) 2007-075023
Apr. 27, 2007 (JP) 2007-118423
May 18, 2007 (JP) 2007-132488
Dec. 4, 2007 (JP) 2007-313047
Dec. 4, 2007 (JP) 2007-313048

(51) Int. Cl. C12Q 1/32; C12M 1/00

(52) U.S. Cl. 435/26; 435/287.9

(57) ABSTRACT

A method of quantifying glucose in a solution characterized in that electric potential measurement is conducted by potentiometry using a glucose dehydrogenase that requires a flavin compound as a coenzyme. It is preferable to carry out the quantification using a glucose dehydrogenase derived from a filamentous fungus, in particular derived from Aspergillus oryzae or Aspergillus terreus.

### BACKGROUND OF THE INVENTION

1. Field of the Invention

The present invention relates to a method for quantifying glucose in a solution by electrochemical means. More specifically, the present invention relates to a method of measuring a glucose amount by measuring an electronic potential between electrodes in a solution using a glucose dehydrogenase that requires a flavin compound as a coenzyme, and a glucose dehydrogenase composition for measuring a glucose amount.

The present invention also relates to an electrochemical sensor for quantifying glucose by electrochemical means. More specifically, the present invention relates to an electrochemical sensor for glucose measurement in which a glucose dehydrogenase is covalently immobilized on a metal electrode.

2. Description of Related Art

The most widely known object of rapid measurement of glucose is measurement of a blood glucose level in a diabetic patient. Furthermore, also in the general industrial world, a technique for rapidly and conveniently measuring a glucose amount is desired in the field of food industry or the like for the purpose of quality control or the like.

In recent years, the incidence rate of diabetes tends to increase year by year. The number of domestic patients including latent persons called reserves is said to be ten million or more. Furthermore, interest in lifestyle-related diseases is increasing very much. Thus, opportunity for and need of careful self measurement of blood glucose levels are increasing. In such historical background, development of techniques for self-monitoring of blood glucose (SMBG) is important for a diabetic patient to grasp the usual blood glucose level and to utilize it for therapy. Regarding techniques for measuring blood glucose, many methods have been reported and put into practice. A method by electrochemical sensing is advantageous as the basic technique for SMBG in view of the small test sample amount, the short measurement time and the small device size.

A large number of sensor techniques each utilizing an enzyme that uses glucose as its substrate are known as sensing techniques for blood glucose measurement which are becoming generally established. Such an enzyme is exemplified by a glucose oxidase (EC 1.1.3.4). Since a glucose oxidase has advantages of the high specificity for glucose and the excellent thermostability, it has been utilized as an enzyme for a blood glucose sensor for a long time. Indeed, the first report was made about 40 yeas ago. Measurement using a blood glucose sensor that utilizes a glucose oxidase is based on the transfer of an electron, which is generated during a course of conversion of glucose into D-glucono-δ-lactone by oxidization, to an electrode via a mediator (electron acceptor). Since the glucose oxidase tends to transfer a proton generated upon the reaction to oxygen, there has been a problem that dissolved oxygen influences the measured value.

For avoiding such a problem, for example, an NAD(P)-dependent glucose dehydrogenase (EC 1.1.1.47) or a pyrroloquinoline quinone (PQQ)-dependent glucose dehydrogenase (EC 1.1.5.2 (formerly EC 1.1.99.17)) has been used as an enzyme for a blood glucose sensor (JP-A 2004-512047 and JP-A 2000-171428). They are advantageous in that they are not influenced by dissolved oxygen and the reactions are rapid. However, the former one, NAD(P)-dependent glucose dehydrogenase, has drawbacks such as the poor stability and the complicatedness due to the required addition of a coenzyme. The latter one, pyrroloquinoline quinone-dependent glucose dehydrogenase has a drawback that since it acts also on saccharides other than glucose such as maltose and lactose due to its poor substrate specificity, the accuracy of the measured value may be lowered. Then, attention has become paid to a flavin adenine dinucleotide (FAD)-dependent glucose dehydrogenase.

A flavin-linked glucose dehydrogenase derived from the genus Aspergillus (flavin-linked glucose dehydrogenase is also referred to as FADGDH herein) is disclosed in WO 2004/058958. This enzyme is advantageous in that it has excellent substrate specificity and it is not influence by dissolved oxygen. As to the thermostability, the activity remaining ratio after treatment at 50° C. for 15 minutes is about 89%. Thus, it is said that the enzyme has excellent stability. Furthermore, a method in which an FADGDH is immobilized on a glassy carbon electrode and glucose is measured by electric current measurement has been disclosed. Although such an electrochemical measurement method using an enzyme electrode is generally and widely used, the fact that it is very difficult to control the enzyme immobilization state is the greatest obstacle. Thus, there is a problem that it is difficult to reproducibly obtain data.

### SUMMARY OF THE INVENTION

The main object of the present invention is to provide a method of quantifying glucose with which stable data can be conveniently and reproducibly obtained by a sensor technique that uses a glucose dehydrogenase that requires a flavin compound as a coenzyme.

As a result of intensive studies, the present inventors have found that the above-mentioned problems can be solved by the means as described below, and attained the present invention. The constitution of the present invention is as follows.

(1) A method of quantifying glucose in a solution, comprising measuring an electric potential by potentiometry using a glucose dehydrogenase that requires a flavin compound as a coenzyme.

(2) The method of quantifying glucose according to (1), wherein the glucose dehydrogenase is a protein of (a) or (b) below:

(a) a protein consisting of the amino acid sequence of SEQ ID NO:1;

(b) a protein having a glucose dehydrogenase activity and consisting of an amino acid sequence in which one or several amino acid(s) is(are) delete, substituted or added in the amino acid sequence of SEQ ID NO:1.

(3) The method of quantifying glucose according to (1), wherein the glucose dehydrogenase is a protein of (c) or (d) below:

(c) a protein consisting of the amino acid sequence of SEQ ID NO:2;

(d) a protein having a glucose dehydrogenase activity and consisting of an amino acid sequence in which one or several amino acid(s) is(are) delete, substituted or added in the amino acid sequence of SEQ ID NO:2.

(4) The method of quantifying glucose according to (1), wherein the glucose dehydrogenase has an amino acid substitution at at least one position in SEQ ID NO:2 selected from the group consisting of position 120, position 160, position 162, position 163, position 164, position 165, position 166, position 167, position 169, position 170, position 171, position 172, position 180, position 329, position 331, position 369, position 471 and position 551.

(5) The method of quantifying glucose according to (4), wherein the glucose dehydrogenase has at least an amino acid substitution of any one of the following in SEQ ID NO:2: K120E, G160E, G160I, G160P, G160S, G160Q, S162A, S162C, S162D, S162E, S162F, S162H, S162L, S162P, G163D, G163K, G163L, G163R, S164F, S164T, S164Y, L165A, L165I, L165N, L165P, L165V, A166C, A166I, A166K, A166L, A166M, A166P, A166S, S167A, S167P, S167R, S167V, N169K, N169P, N169Y, N169W, L170C, L170F, S171I, S171K, S171M, S171Q, S171V, V172A, V172C, V172E, V172I, V172M, V172S, V172W, V172Y, A180G, V329Q, A331C, A331D, A331I, A331K, A331L, A331M, A331V, K369R, K471R, V551A, V551C, V551T, V551Q, V551S, V551Y, (G160E+S167P), (G160I+S167P), (G160S+S167P), (G160Q+S167P) , (S162A+S167P) , (S162C+S167P) , (S162D+S167P) (S162D+S167P), (S162E+S167P), (S162F+S167P), (S162H+S167P), (S162L+S167P), (G163D+S167P), (S164F+S167P), (S164T+S167P), (S164Y+S167P), (L165A+S167P), (L165I+S167P), (L165P+S171K), (L165P+V551C), (L165V+V551C), (A166C+S167P), (A166I+S167P), (A166K+S167P), (A166K+S167P), (A166M+S167P), (A166P+S167P), (A166S+S167P), (S167P+N169K), (S167P+N169P), (S167P+N169Y), (S167P+N169W), (S167P+L170C), (S167P+L170F), (S167P+S171I), (S167P+S171K), (S167P+S171M), (S167P+S171Q), (S167P+S171V), (S167P+V172A), (S167P+V172C), (S167P+V172E), (S167P+V172I), (S167P+V172M), (S167P+V172S), (S167P+V172T), (S167P+V172W), (S167P+V172Y), (S167P+V329Q), (S167P+A331C), (S167P+A331D), (S167P+A331I), (S167P+A331K), (S167P+A331L), (S167P+A331M), (S167P+A331V), (G163K+V551C), (G163R+V551C).

(6) The method of quantifying glucose according to (1), wherein the glucose dehydrogenase has an amino acid substitution at at least one position in SEQ ID NO:2 selected from the group consisting of position 163, position 167 and position 551.

(7) The method of quantifying glucose according to (6), wherein the glucose dehydrogenase has at least an amino acid substitution of any one of the following in SEQ ID NO:2: S167P, V551C, (G163K+V551C) and (G163R+V551C).

(8) The method of quantifying glucose according to (1), wherein the glucose dehydrogenase exhibits an activity remaining ratio of 20% or more after heating at 50° C. for 15 minutes.

(9) The method of quantifying glucose according to (1), wherein the glucose dehydrogenase exhibits a remaining activity of 80% or more after treatment at pH 4.5 to pH 6.5 at 25° C. for 16 hours.

(10) The method of quantifying glucose according to (1), wherein the glucose dehydrogenase is derived from a filamentous fungus.

(11) The method of quantifying glucose according to (10), wherein filamentous fungus belongs to the genus Penicillium or the genus Aspergillus.

(12) The method of quantifying glucose according to (11), wherein the filamentous fungus belongs to Aspergillus oryzae.

(13) The method of quantifying glucose according to (1), wherein a glucose reaction is detected by measuring a liquid junction potential in a solution of the glucose dehydrogenase that requires a flavin compound as a coenzyme, using a printed electrode in which a metal electrode is formed on an insulated substrate.

(14) The method of quantifying glucose according to (13), wherein the detection of the glucose reaction is mediated by an electron transfer by a mediator.

(15) An enzymatic reaction composition for measuring an electric potential by potentiometry, wherein a glucose dehydrogenase that requires a flavin compound as a coenzyme contained in the composition complies with one or more of the following:

(1) being dissolved in a Good's buffer

(2) coexisting with at least one compound selected from the group consisting of triethanolamine, Tricine, imidazole and collidine; and

(3) coexisting with a halogen compound.

(16) The enzymatic reaction composition according to (15), wherein the Good's buffer is one or more selected from the group consisting of MOPS, PIPES, HEPES, MES, TES, BES, ADA, POPSO, Bis-Tris, Bicine, Tricine, TAPS, CAPS, EPPS, CAPSO, CHES, MOPSO, DIPSO, TAPS, TAPSO and HEPPSO.

(17) The enzymatic reaction composition according to (15), wherein the glucose dehydrogenase coexists with as the halogen compound at least one compound selected from the group consisting of iodoacetic acid, iodoacetamide and sodium fluoride.

(18) The enzymatic reaction composition according to (15), wherein the glucose dehydrogenase is a protein of (a) or (b) below:

(a) a protein consisting of the amino acid sequence of SEQ ID NO:1;

(b) a protein having a glucose dehydrogenase activity and consisting of an amino acid sequence in which one or several amino acid(s) is(are) delete, substituted or added in the amino acid sequence of SEQ ID NO:1.

(19) The enzymatic reaction composition according to (15), wherein the glucose dehydrogenase is a protein of (c) or (d) below:

(c) a protein consisting of the amino acid sequence of SEQ ID NO:2;

(d) a protein having a glucose dehydrogenase activity and consisting of an amino acid sequence in which one or several amino acid(s) is(are) delete, substituted or added in the amino acid sequence of SEQ ID NO:2.

(20) An electrochemical sensor for glucose measurement, in which a glucose dehydrogenase is covalently immobilized on a metal electrode via an alkanethiol or a hydrophilic macromolecule, and with which a glucose reaction is detected electrochemically.

(21) The electrochemical sensor for glucose measurement according to (20), wherein the metal electrode is formed on an insulated substrate.

(22) The electrochemical sensor for glucose measurement according to (20), wherein the metal electrode is round-shaped.

(23) The electrochemical sensor for glucose measurement according to (22), wherein the radius of the metal electrode is 2 mm or less.

(24) The electrochemical sensor for glucose measurement according to (20), wherein the hydrophilic macromolecule is polyethylene glycol (PEG).

(25) The electrochemical sensor for glucose measurement according to (20), wherein a change in an electric current generated due to the action with glucose is measured.

(26) The method of quantifying glucose according to (1), wherein the activity of the glucose dehydrogenase is inhibited in the presence of 1 mM 1,10-phenanthroline by 30% or more.

(27) The method of quantifying glucose according to (26), wherein the glucose dehydrogenase is a protein of (a) or (b) below:

(a) a protein consisting of the amino acid sequence of SEQ ID NO:3;

(b) a protein having a glucose dehydrogenase activity and consisting of an amino acid sequence in which one or several amino acid(s) is(are) delete, substituted or added in the amino acid sequence of SEQ ID NO:3.

(28) The method of quantifying glucose according to (26), wherein the glucose dehydrogenase is derived from the genus Penicillium or the genus Aspergillus.

(29) The method of quantifying glucose according to (26), wherein the glucose dehydrogenase is derived from Aspergillus terreus.

A concentration of glucose in a solution can be conveniently and reproducibly quantified by using the method according to the present invention. It is expected to be very useful for food analysis in addition to application to a blood glucose sensor, of course.

### BRIEF DESCRIPTION OF THE SEVERAL VIEWS OF THE DRAWINGS

FIG. 1 illustrates a scheme of chemical reactions for glucose sensing using a glucose dehydrogenase.

FIG. 2 illustrates measurement results of changes in electric potentials due to the addition of glucose measured in Example 1.

FIG. 3 illustrates a calibration curve of electric potentials in the steady state versus glucose concentrations measured in Example 1.

FIG. 4 illustrates results of reproducibility examination of measurement results in Example 1.

FIG. 5 illustrates measurement results of changes in electric potentials due to the addition of glucose measured in Example 2.

FIG. 6 illustrates calibration curves of electric potentials in the steady state versus glucose concentrations measured in Example 2 which were obtained using: (A) 100 mM PIPES buffer (pH 7.0); and (B) 100 mM Tris-hydrochloride buffer (pH 7.0).

FIG. 7 illustrates results of reproducibility examination of measurement results in Example 2.

FIG. 8 illustrates exemplary measurement results of changes in electric potentials due to the addition of glucose measured in Example 3.

FIG. 9 illustrates calibration curves of electric potentials in the steady state versus glucose concentrations measured in the presence of various additives in Example 3.

FIG. 10 illustrates results of examination of reaction selectivity with xylose in the presence of various additives in Example 4.

FIG. 11 illustrates exemplary measurement results of changes in electric potentials due to the addition of glucose measured in Example 5.

FIG. 12 illustrates changes in electric potentials measured in the presence of various additives in Example 5.

FIG. 13 illustrates measurement results of changes in electric potentials due to the addition of glucose measured in Example 6.

FIG. 14 illustrates calibration curves of electric potentials in the steady state versus glucose concentrations measured in Example 6.

FIG. 15 illustrates results of reproducibility examination of measurement results in Example 6.

FIG. 16 illustrates the electrode used in Examples 7 to 9.

FIG. 17 illustrates a graph in which the relationship between the ratio of the potassium ferrocyanide solution/the potassium ferricyanide solution and the electric potential is plotted in Example 7.

FIG. 18 illustrates the results of potentiometry in Example 8.

FIG. 19 illustrates a graph in which the relationship between the glucose concentration and the electric potential is plotted obtained by conducting potentiometry in Example 9.

FIG. 20 illustrates (A) the electrode used in Examples 10 and 11; and (B) the state of a gold electrode mounted with a solution in Examples 10 and 11.

FIG. 21 illustrates the results of amperometry in Example 10.

FIG. 22 illustrates the results of amperometry in Example 11.

### DETAILED DESCRIPTION OF THE INVENTION

The present invention is characterized in that a glucose dehydrogenase that requires a flavin compound as a coenzyme is used. Flavins are a group of derivatives each having a substituent at the 10-position of dimethyl isoalloxazine. There is no specific limitation as long as the enzyme uses a flavin molecular species as a coenzyme. Examples of flavin compounds include flavin adenine dinucleotide (FAD) and flavin adenine mononucleotide (FMN). FAD is particularly preferable.

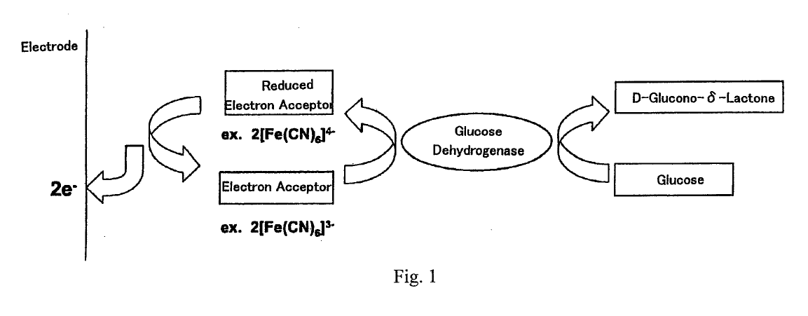

A glucose dehydrogenase catalyzes a reaction of oxidizing a hydroxy group of glucose to generate glucono-δ-lactone in the presence of a mediator (electron acceptor). A scheme of sensing is shown in FIG. 1. When a FAD-dependent glucose dehydrogenase acts on glucose, the coenzyme FAD is converted into FADH₂. In the presence of a ferricyanide (e.g., [Fe(CN)₆]³⁻) as a mediator, FADH₂ converts it into a ferrocyanide ([Fe(CN)₆]⁴⁻ in this case), and returns to FAD. When an electric potential is applied to a ferrocyanide, the ferrocyanide transfers an electron to an electrode, and returns to a ferricyanide. Then, use of such an electron carrier as a mediator enables electrochemical signal detection.

Although there is no specific limitation concerning the origin of a glucose dehydrogenase used according to the present invention, one derived from a filamentous fungus is preferable. In particular, it is exemplified by one derived from a fungus belonging to the genus Penicillium or the genus Aspergillus. One derived from the genus Aspergillus is particularly preferable, and one derived from Aspergillus oryzae is still more preferable. A glucose dehydrogenase may be a naturally-occurring one obtained by extraction and purification, or one produced using known genetic engineering techniques based on genetic information.

Specific examples of glucose dehydrogenases used according to the present invention include one consisting of the amino acid sequence of SEQ ID NO:1 (593 amino acid residues). The amino acid sequence is not limited only to one that completely matches SEQ ID NO:1. A protein having a glucose dehydrogenase activity and having an amino acid sequence in which one or several amino acid(s) is(are) deleted, substituted or added is also encompassed.

A glucose dehydrogenase that has excellent thermostability is particularly preferably used according to the present invention. Specific example thereof is one that exhibits an activity remaining ratio of 20% or more after heating at 50° C. for 15 minutes. Preferably, it exhibits an activity remaining ratio of 40% or more after heating at 50° C. for 15 minutes. More preferably, it exhibits an activity remaining ratio of 80% or more after heating at 50° C. for 15 minutes.

A glucose dehydrogenase that has excellent pH stability is particularly preferably used according to the present invention. Specific example thereof is one that exhibits a remaining activity of 80% or more after treatment at pH 4.5 to pH 6.5 at 25° C. for 16 hours. Preferably, it exhibits a remaining activity of 90% or more after treatment at pH 4.5 to pH 6.5 at 25° C. for 16 hours.

For conferring the thermostability or pH stability as described above, it is also preferable to introduce a mutation using genetic engineering techniques based on the amino acid sequence of SEQ ID NO:1. For example, the amino acid sequence of SEQ ID NO:2 in which the signal peptide is cleaved from SEQ ID NO:1 may be used. A mutation can be introduced applying a known method. Specifically, a DNA having genetic information for a protein with modification is prepared by altering, or inserting or deleting, a certain nucleotide in a DNA having genetic information for the protein. In a specific exemplary method for altering a nucleotide sequence of a DNA, a commercially available kit (Transformer Mutagenesis Kit (Clonetech), EXOIII/Mung Bean Deletion Kit (Stratagene), Quick Change Site Directed Mutagenesis Kit (Stratagene), etc.) is used, or polymerase chain reaction (PCR) is utilized.

For example, a glucose dehydrogenase with increased thermostability is exemplified by one having an amino acid substitution at at least one position in the amino acid sequence of SEQ ID NO:2 selected from the group consisting of position 120, position 160, position 162, position 163, position 164, position 165, position 166, position 167, position 169, position 170, position 171, position 172, position 180, position 329, position 331, position 369, position 471 and position 551. Further, it is exemplified by one having an amino acid substitution at at least one of position 162, position 163, position 167 and position 551 among the above-mentioned positions.

More specifically, it is exemplified by one having an amino acid substitution selected from the group consisting of the following in the amino acid sequence of SEQ ID NO:2: K120E, G160E, G160I, G160P, G160S, G160Q, S162A, S162C, S162D, S162E, S162F, S162H, S162L, S162P, G163D, G163K, G163L, G163R, S164F, S164T, S164Y, L165A, L165I, L165N, L165P, L165V, A166C, A166I, A166K, A166L, A166M, A166P, A166S, S167A, S167P, S167R, S167V, N169K, N169P, N169Y, N169W, L170C, L170F, S171I, S171K, S171M, S171Q, S171V, V172A, V172C, V172E, V172I, V172M, V172S, V172W, V172Y, A180G, V329Q, A331C, A331D, A331I, A331K, A331L, A331M, A331V, K369R, K471R, V551A, V551C, V551T, V551Q, V551S and V551Y. “K120E” means replacement of K (Lys) at position 120 with E (Glu).

In particular, a preferable embodiment is exemplified by an amino acid substitution of G163K, G163L, G163R, S167P, V551A, V551C, V551Q, V551S, V551Y, (G160I+S167P) , (S162F+S167P) , (S167P+N169Y) , (S167P+L171I), (S167P+L171K), (S167P+L171V), (S167P+V172I), (S167P+V172W), (G163K+V551C) or (G163R+V551C).

Furthermore, a glucose dehydrogenase having increased pH stability is exemplified by one having an amino acid substitution at at least one position in the amino acid sequence of SEQ ID NO:2 selected from the group consisting of position 163, position 167 and position 551. More specifically, it is exemplified by one having at least an amino acid substitution of any one of the following in SEQ ID NO:2: S167P, V551C, (G163K+V551C) and (G163R+V551C).

The sequence that serves as the basis for mutagenesis of a glucose dehydrogenase used according to the present invention is not limited to one that completely matches the amino acid sequence of SEQ ID NO:2. The sequence encompasses one having a mutation at a position of identity observed upon alignment in homology analysis with another Aspergillus oryzae-derived glucose dehydrogenase having an amino acid sequence highly homologous to the amino acid sequence of SEQ ID NO:2. Specifically, homology of preferably 80% or more, more preferably 85% or more, still more preferably 90% or more is exhibited in exemplary cases.

According to the present invention, choice of the type of the solution used for the enzymatic reaction is very important for the reactivity or stability of the glucose dehydrogenase.

The glucose dehydrogenase used according to the present invention may comply with one or more of the following:

(1) being dissolved in a Good's buffer

(2) coexisting with at least one compound selected from the group consisting of triethanolamine, Tricine, imidazole and collidine; and

(3) coexisting with a halogen compound.

Choice of the type of the solution used for the enzymatic reaction is very important for the reactivity or stability of the glucose dehydrogenase. The present invention is characterized in that a Good's buffer is used in particular. The Good's buffers are frequently used in the field of biochemistry, and use various derivatives of aminoethanesulfonic acid or aminopropanesulfonic acid each having a zwitterion structure. The characteristics are as follows.

(1) It is dissolved in water well; a thick buffer can be prepared.

(2) It permeates little through a biological membrane.

(3) The acid dissociation equilibrium is influenced little by the concentration, the temperature or the ionic composition.

(4) The ability to form a complex with a metal ion is low.

(5) It is chemically stable, and can be highly purified by recrystallization.

(6) It can be used to readily detect the component of interest due to the lack of visible or ultraviolet absorption.

Examples of types of Good's buffers include MOPS, PIPES, HEPES, MES, TES, BES, ADA, POPSO, Bis-Tris, Bicine, Tricine, TAPS, CAPS, EPPS, CAPSO, CHES, MOPSO, DIPSO, TAPS, TAPSO and HEPPSO. Although there is no specific limitation concerning the type of Good's buffer, PIPES is particularly preferable. The pH of the buffer is preferably about 4.0 to 9.0, more preferably about 5.0 to 8.0, still more preferably about 5.5 to 7.5. The concentration is preferably about 1 to 200 mM, more preferably about 10 to 150 mM, still more preferably about 20 to 100 mM. Furthermore, succinic acid, maleic acid, malic acid, phthalic acid, imidazole, triethanolamine, collidine, a salt thereof or the like may optionally coexist in an enzymatic reaction mixture for increasing the storage stability. A saccharide may also be added.

The present invention is characterized in that a glucose dehydrogenase coexists in a reaction composition with at least one compound selected from the group consisting of triethanolamine, Tricine, imidazole and collidine. The coexistence with such a compound increases the quantitativeness and the substrate selectivity. Furthermore, there also is an effect of increasing the substrate specificity of the glucose dehydrogenase, in particular, the reaction selectivity for xylose. The addition may be carried out alone or in combination.

Although there is no specific limitation concerning the type of the solution used for an enzymatic reaction, examples thereof include phosphate buffers (e.g., PBS) and Good's buffers (e.g., MOPS, PIPES, HEPES, MES and TES). The pH of the buffer is preferably about 4.0 to 9.0, more preferably about 5.0 to 8.0, still more preferably about 5.5 to 7.5. The concentration is preferably about 1 to 200 mM, more preferably about 10 to 150 mM, still more preferably about 20 to 100 mM.

The present invention is characterized in that a glucose dehydrogenase coexists in a reaction composition with a halogen compound. A halogen compound is a compound that contains a halogen atom, i.e., bromine, chlorine, iodine or fluorine. There is no specific limitation concerning the structure thereof. One containing iodine or fluorine as the halogen atom is particularly preferable. Specific examples of such compounds include, but are not limited to, iodoacetic acid (MIA), iodoacetamide (IAA) and sodium fluoride (NaF). The coexistence concentration is about 1 μM to 10 mM, more preferably about 10 μM to 1 mM. The coexistence with such a compound enables increase in the initial velocity of the enzymatic reaction in particular, thus increasing the rapidness of the measurement. The addition may be carried out alone or in combination.

Although there is no specific limitation concerning the type of the solution used for an enzymatic reaction, examples thereof include phosphate buffers and Good's buffers (e.g., MOPS, PIPES, HEPES, MES and TES). The pH of the buffer is preferably about 4.0 to 9.0, more preferably about 5.0 to 8.0, still more preferably about 5.5 to 7.5. The concentration is preferably about 1 to 200 mM, more preferably about 10 to 150 mM, still more preferably about 20 to 100 mM.

The activity of the glucose dehydrogenase that requires a flavin compound as a coenzyme used according to the present invention may be inhibited in the presence of 1 mM 1,10-phenanthroline by 30% or more, preferably 40% or more, more preferably 50% or more. 1,10-Phenanthroline is a condensed aromatic compound which is used as a chelating ligand for a transition metal in many cases. It is important according to the present invention to use a glucose dehydrogenase having a property of being inhibited by 1,10-phenanthroline.

Although there is no specific limitation concerning the origin of the glucose dehydrogenase of which the activity is inhibited in the presence of 1 mM 1,10-phenanthroline by 30% or more, one derived from a filamentous fungus is preferable. In particular, it is exemplified by one derived from a fungus belonging to the genus Penicillium or the genus Aspergillus. One derived from the genus Aspergillus is particularly preferable, and one derived from Aspergillus terreus is still more preferable. A glucose dehydrogenase may be a naturally-occurring one obtained by extraction and purification, or one produced using known genetic engineering techniques based on genetic information.

Specific examples of glucose dehydrogenases of which the activity is inhibited in the presence of 1 mM 1,10-phenanthroline by 30% or more include one consisting of the amino acid sequence of SEQ ID NO:3 (568 amino acid residues). The amino acid sequence is not limited only to one that completely matches SEQ ID NO:3. A protein having a glucose dehydrogenase activity and having an amino acid sequence in which one or several amino acid(s) is(are) deleted, substituted or added is also encompassed.

The sequence that serves as the basis for mutagenesis of the glucose dehydrogenase of which the activity is inhibited in the presence of 1 mM 1,10-phenanthroline by 30% or more is not limited to one that completely matches the amino acid sequence of SEQ ID NO:3. The sequence encompasses one having a mutation at a position of identity observed upon alignment in homology analysis with another Aspergillus terreus-derived glucose dehydrogenase having an amino acid sequence highly homologous to the amino acid sequence of SEQ ID NO:3. Specifically, homology of preferably 80% or more, more preferably 85% or more, still more preferably 90% or more is exhibited in exemplary cases.

The present invention is characterized in that an electric potential is measured particularly by potentiometry among electrochemical measurement techniques. Potentiometry refers to a method in which physicochemical information about a system in which an enzymatic reaction takes place is obtained by placing a working electrode and a reference electrode in a solution, and measuring the difference in electronic potential between the electrodes to determine the electric potential of the working electrode relative to the reference electrode. The relationship between an ion or a molecule in a solution and an electrode potential conforms to the Nernst equation. An ion or a molecule can be identified using this equation. Furthermore, an electrode potential is changed according to the change in the concentration of an ion or a molecule in a solution. Then, it is possible to estimate the concentration of an ion or a molecule based on the measured electric potential value. Since electric potential measurement upon an enzymatic reaction in a solution is sufficient for this method, the system is very simple and the operation is also simple. Furthermore, the method does not require immobilization of an enzyme, which is required for a measurement method using an enzyme electrode. Therefore, time and effort required for the immobilization are unnecessary. In addition, it is not necessary to consider the difficulty in reproducing the immobilization state, and data can be obtained stably and reproducibly. Thus, the method is very useful in these respects.

Although there is no specific limitation concerning the method of measuring an electric potential, a general potentiostat or galvanostat or the like can be used. A general tester may be used. Although the measurement system may be a system of three electrodes, it is usually possible to conduct the measurement using only two electrodes. There is no specific limitation concerning the type of the electrode, and one generally used for electrochemical experiments can be applied. Platinum, gold, glassy carbon, carbon paste, PFC (plastic formed carbon) or the like can be used for the working electrode. A saturated calomel electrode, silver-silver chloride or the like can be used for the reference electrode.

Although there is no specific limitation concerning the solution used for an enzymatic reaction, it is preferable to use a buffer having a composition that is advantageous in view of the reactivity or stability of the glucose dehydrogenase. Examples of types of buffers include phosphate, citrate, acetate, borate, Tris, PIPES and MES. The pH is preferably about 4.0 to 9.0, more preferably about 5.0 to 8.0, still more preferably about 5.5 to 7.5. The concentration is preferably about 1 to 200 mM, more preferably about 10 to 150 mM, still more preferably about 20 to 100 mM. Succinic acid, maleic acid, malic acid, phthalic acid, imidazole, triethanolamine, collidine, a salt thereof or the like may optionally coexist in an enzymatic reaction mixture for increasing the storage stability. A saccharide may also be added.

The electric potential measurement method may be a method in which glucose in a solution is measured by potentiometry using a glucose dehydrogenase, using a printed electrode having a metal electrode.

In the above-mentioned method, a metal such as platinum, gold, nickel or palladium is used for the working electrode. Use of a metal electrode is advantageous particularly in view of the electron transfer velocity.

It is preferable that a metal electrode is formed on an insulated substrate in the printed electrode in the above-mentioned method. Specifically, an electrode is desirably formed on a substrate using a printing technique such as photolithography technique, screen printing, gravure printing or flexographic printing. Materials for the insulated substrates include silicon, glass, ceramic, polyvinyl chloride, polyethylene, polypropylene and polyester. Ones highly resistant to various solvents and chemicals are more preferable. According to the present invention, there is no specific limitation concerning the shape of the metal electrode. It may be round-shaped, elliptic or square.

In the above-mentioned method, it is preferable that the scale of the printed electrode is as small as possible. The area of the metal electrode as the working electrode is preferably about 3 to 5 mm². If the working electrode is round-shaped, the radius is preferably 3 mm or less, more preferably 2.5 mm or less, still more preferably 2 mm or less.

The present invention also relates to an electrochemical sensor, in which a glucose dehydrogenase is covalently immobilized on a metal electrode via an alkanethiol or a hydrophilic macromolecule, and with which a reaction of glucose (which is the substrate for the immobilized glucose dehydrogenase) is detected electrochemically. There is no specific limitation concerning the method for electrochemical detection. In an exemplary method, a change in electric current generated due to an oxidation or reduction reaction upon the action with the substrate is measured by amperometry.

Although there is no specific limitation concerning the method of electrochemical measurement for the sensor, a general potentiostat or galvanostat or the like can be used. A general tester may be used. The measurement system may be a system of two electrodes or three electrodes. According to the present invention, a metal such as platinum, gold, silver, nickel or palladium is used for the working electrode. Among these, gold is particularly preferable. Although it is not intended to limit the present invention, one generally used for electrochemical experiments can be applied to the reference electrode. For example, saturated calomel electrode, silver-silver chloride or the like can be used.

A thiol group is known to specifically react with a metal to form a self-assembled monolayer (SAM). When a metal is used for a working electrode, it is possible to readily introduce a functional group onto the surface applying this principle. Thus, it is possible to immobilize an enzyme on the surface of the electrode. This immobilization method is favorable for the reproducibility because the enzyme immobilization density can be readily controlled particularly as compared with immobilization of an enzyme by physical adsorption. A metal electrode is advantageous to handling in that SAM formation can be readily accomplished as described above.

According to the present invention, a glucose dehydrogenase is covalently immobilized on a metal electrode via an alkanethiol or a hydrophilic macromolecule among others. Due to this constitution, it is possible not only to increase the stability of the enzyme, but also to increase the reactivity with the substrate. Then, it is possible to realize highly accurate measurement.

If an alkanethiol is to be used, one having a thiol group at the end of an alkyl chain of about 3 to 20 carbons is preferable. Furthermore, one in which a functional group for immobilizing an enzyme is introduced at the other end is preferable. The functional groups include a carboxyl group, a thiol group, an aldehyde group and a succinimide group. For example, if an alkanethiol which has both a thiol group and a functional group is used as a crosslinker, it is possible to introduce a functional group onto a surface of a metal electrode in a single step.

As used herein, a hydrophilic macromolecule refers to a compound having a property of being soluble in water or swelling in water, and having a repeating unit. It may be a synthetic one or a naturally-occurring one. Specific examples of the hydrophilic macromolecules include: polyethylene glycol (PEG); polyvinyl alcohol; polymethacrylic acid; polymethacrylate; polymethacrylamide; polyethylene imine; polyvinylpyrrolidone; polyester or polyurethane in which a hydrophilic moiety such as a monomer or polyethylene glycol containing carboxylic acid or a salt thereof, or sulfonic acid or a salt thereof is copolymerized; carboxymethylcellulose; and polysaccharides such as chitosan, carrageenan and glucomannna. Among these, ones that do not have a reactive moiety such as an OH group, carboxylic acid or a salt thereof, amine or imine (e.g., polyethylene glycol, polymethacrylamide and polyvinylpyrrolidone) are preferable. PEG is most preferable.

For example, if an enzyme is to be immobilized using PEG as such a hydrophilic macromolecule, it is possible to use a PEG derivative that is modified with a functional group or the like. Particularly when a metal electrode is used, it is preferable to use a PEG derivative having a thiol group at the end. Furthermore, it is preferable that the PEG has, at the other end, a functional group that is capable of coupling an enzyme which is a protein. Such functional groups include a carboxyl group, a thiol group, an aldehyde group and a succinimide group. For example, if a PEG derivative having both a thiol group and a succinimide group is used as a crosslinker, it is possible to introduce a succinimide group onto a surface of a metal electrode in a single step. Alternatively, a PEG derivative having both a thiol group and a carboxyl group may be used as a crosslinker to conduct immobilization by a condensation reaction using a water-soluble carbodiimide. In such cases, the repetition of ethylene glycol in the PEG crosslinker is preferably about 3 to 25.

In case of a thiol group which is relatively unstable, a metal electrode may be subjected to surface treatment by subjecting a crosslinker (preferably, a crosslinker consisting of a PEG derivative) in which the thiol group is protected to simple chemical treatment to form a thiol group upon use. Specifically, a compound having an S-acetyl group at the end may be used. It is possible to form a thiol group by subjecting it to deacetylation. More preferably, a PEG derivative having both an S-acetyl group and a succinimide group is used.

According to the present invention, it is preferable that a metal electrode is formed on an insulated substrate. Specifically, an electrode is desirably formed on a substrate using a printing technique such as photolithography technique, screen printing, gravure printing or flexographic printing. Materials for the insulated substrates include silicon, glass, ceramic, polyvinyl chloride, polyethylene, polypropylene and polyester. Ones highly resistant to various solvents and chemicals are more preferable.

According to the present invention, there is no specific limitation concerning the shape of the metal electrode. It may be round-shaped, elliptic or square. In particular, it is preferably round-shaped in view of easy mounting of a solution of an enzyme to be immobilized. If it is round-shaped, the radius is preferably 3 mm or less, more preferably 2.5 mm or less, still more preferably 2 mm or less. The volume of the enzyme solution of about 1 to 5 μl is sufficient, and the volume is more preferably about 2 to 3 μl. An immobilization reaction after mounting an enzyme solution is preferably conducted by standing under humid conditions.

According to the present invention, it is also effective to use a mediator (electron acceptor) for mediating electron transfer between the enzymatic reaction and the electrode. There is no specific limitation concerning the type of the mediator that can be applied. Examples thereof include ferricyanide compounds, phenazine methosulfate, 1-methoxy-5-methylphenazium methylsulfate and 2,6-dichlorophenolindophenol. Examples of electron pairs as another expression include benzoquinone/hydroquinone, ferricyanide/ferrocyanide and ferricinium/ferrocene. Phenazine methosulfate, 1-methoxy-5-methylphenazium methylsulfate or 2,6-dichlorophenolindophenol may be used. Furthermore, various complexes containing a compound other than iron can be used. For example, a metal complex such as osmium or ruthenium can be used. If a compound with low water solubility is to be used as a mediator, use of an organic solvent may impair the stability of the enzyme or inactivate the enzyme. Then, one modified with a hydrophilic macromolecule (e.g., PEG) for increasing the water solubility may be used. The concentration of a mediator in a reaction system is within a range of preferably about 1 mM to 1 M, more preferably 5 mM to 500 mM, still more preferably 10 mM to 300 mM. Also, a mediator modified with one of various functional groups may be used to immobilize it on a metal electrode along with an enzyme.

Upon an enzymatic reaction, measurement is initiated at the time of adding a given amount of a sample solution containing a substrate to a desired volume of a reaction mixture in which desired amounts of an enzyme and a mediator are added and mixed. Although it is not intended to limit the present invention, in electrochemical detection, it is preferable to conduct the measurement using, as a signal, a change in electric current generated as a result of transfer of an electron mediated by a mediator as the enzymatic reaction proceeds. As the enzymatic reaction proceeds, the electric potential begins to decline and becomes steady after a while. The electric potential value in the steady state varies depending on the glucose amount. There is no specific limitation concerning the type of the sample to be subjected to measurement. It may be an aqueous solution that contains, or may contain, the substrate for the enzyme as its component, or a biological sample such as blood, body fluid or urine. Upon measurement, it is preferable to conduct the enzymatic reaction while slowly stirring using a stirrer or the like. It is preferable to make the reaction temperature as constant as possible. It is preferable to use an enzyme that makes the electric potential steady as rapidly as possible. Furthermore, development of microanalysis using a microfluidic device or the like is also possible.

### EXAMPLES

The following Examples illustrate the present invention in more detail, but are not to be construed to limit the scope thereof.

### Example 1

200 μl of a 500 mM potassium ferricyanide solution (final concentration of 100 mM) and 11.4 μl of 10.6 kU/ml FAD-dependent glucose dehydrogenase (corresponding to 120 U) were added to a 2-ml water-jacketed glass cell (BAS). A 100 mM phosphate buffer (pH 7.0) was further added thereto to result in a total volume of 1.2 ml including the volume of a 1 M glucose solution to be added later. The mixture was slowly stirred using a stirrer. One having the mutation of G163R+V551C in SEQ ID NO:2 was used as the FAD-dependent glucose dehydrogenase. The temperature was made constant at 30° C. during the enzymatic reaction by circulating water maintained at 30° C. in a thermostat bath through the water jacket part.

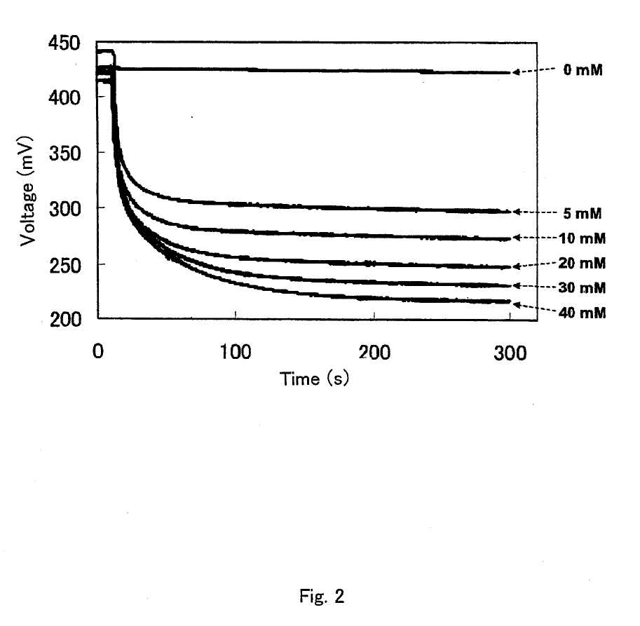

PTE platinum electrode (6.0×1.6 mm; BAS) was used as a working electrode, and RE-1C saturated KCl silver-silver chloride reference electrode (BAS) was used as a reference electrode. These electrodes were placed so that they were immersed in the above-mentioned solution and connected to a general-purpose electrochemical measurement apparatus potentio/galvanostat model 1112 (Fuso Seisakusho) Then, 6, 12, 24, 36 or 48 μl of a 1 M glucose solution was added thereto, and the electric potential value was immediately measured over time in each case. The glucose concentrations in the reaction systems were 5, 10, 20, 30 and 40 mM, respectively. Blank measurement was also conducted without the addition of glucose. The measurement results are shown in FIG. 2. It was observed that as the enzymatic reaction proceeded, the electric potential declined over time and became steady after a while. It was confirmed that the increased amount of glucose added as a substrate resulted in the greater decline in electric potential.

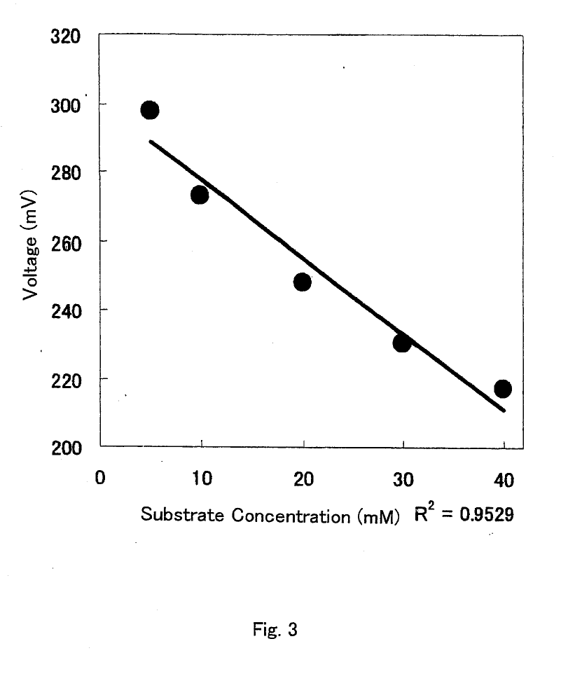

A calibration curve was prepared by plotting the electric potential values in the steady state in FIG. 2 against the glucose concentrations. The results are shown in FIG. 3. The electric potential value observed 300 seconds after the initiation of reaction was plotted along the longitudinal axis. A correlation coefficient R²=0.9529 was obtained upon regression calculation, and very good correlation was exhibited within a wide concentration range up to 40 mM. It was suggested that the amount of glucose in the solution could be accurately quantified by measuring the electric potential in the steady state according to the method of the present invention.

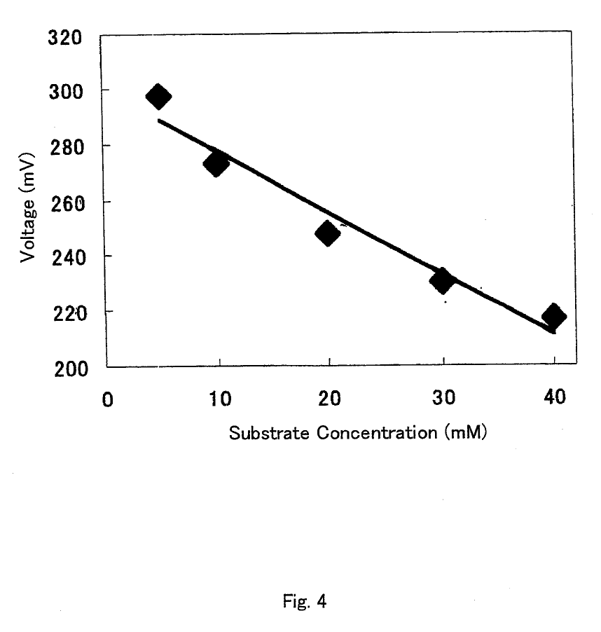

Furthermore, the reproducibility of the above-mentioned measurement was examined. The above-mentioned measurement was conducted once more. The results are shown in FIG. 4. As a result, a calibration curve almost identical to that in FIG. 3 was obtained. Thus, it was confirmed that the reproducibility was very excellent.

The same FAD-dependent glucose dehydrogenase was immobilized on a carbon electrode, electric current measurement was conducted by amperometry at various glucose concentrations, and trial calibration was conducted. However, highly reliable results could not be obtained in this case due to the poor reproducibility of the data.

### Example 2

An enzymatic reaction was conducted as described in Example 1 using a 100 mM PIPES buffer (pH 7.0) in place of the 100 mM phosphate buffer (pH 7.0).

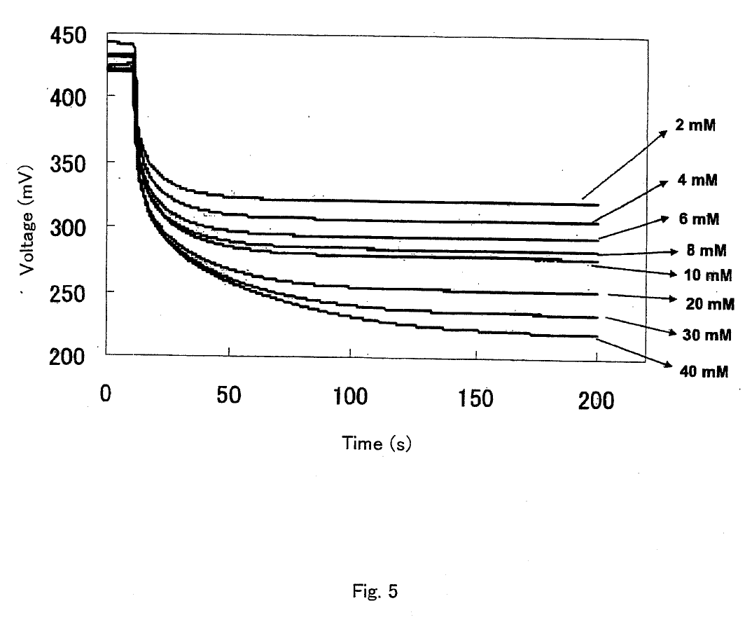

A working electrode and a reference electrode were set as described in Example 1 and connected to the general-purpose electrochemical measurement apparatus. Then, 2.4, 4.8, 7.2, 9.6, 12, 24, 36 or 48 μl of a 1 M glucose solution was added thereto, and the electric potential value was immediately measured over time in each case. The glucose concentrations in the reaction systems were 2, 4, 6, 8, 10, 20, 30 and 40 mM, respectively. Blank measurement was also conducted without the addition of glucose. The measurement results are shown in FIG. 5. It was observed that as the enzymatic reaction proceeded, the electric potential declined over time and became steady after a while. It was confirmed that the increased amount of glucose added as a substrate resulted in the greater decline in electric potential.

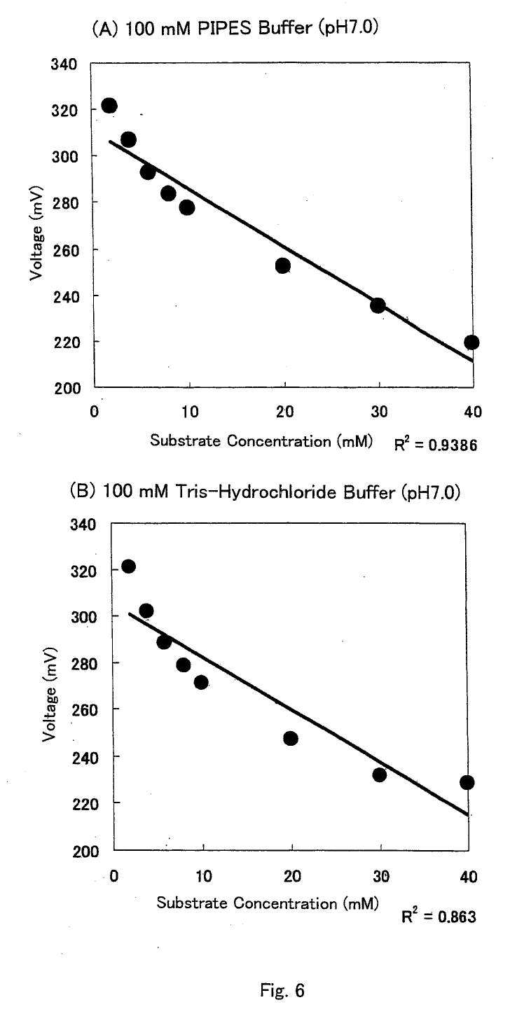

A calibration curve was prepared by plotting the electric potential values in the steady state in FIG. 5 against the glucose concentrations. The results are shown in FIG. 6(A). The electric potential value observed 200 seconds after the initiation of reaction was plotted along the longitudinal axis. A correlation coefficient R²=0.9386 was obtained upon regression calculation, and very good correlation was exhibited within a wide concentration range up to 40 mM. It was suggested that the amount of glucose in the solution could be accurately quantified by measuring the electric potential in the steady state according to the method of the present invention.

Furthermore, examination was conducted under the same conditions as those of Example 1 except that 100 mM Tris-hydrochloride buffer (pH 7.0) was used in place of 100 mM PIPES buffer (pH 7.0). The obtained calibration curve is shown in FIG. 6(B). In this case, the observed linearity was not so good with a correlation coefficient R²=0.863.

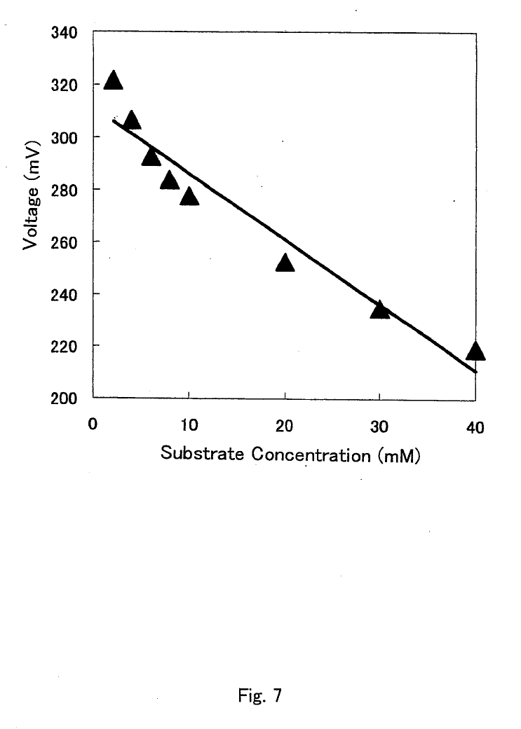

Furthermore, the reproducibility of the above-mentioned measurement was examined. The above-mentioned measurement was conducted once more. The results are shown in FIG. 7. As a result, a calibration curve almost identical to that in FIG. 6 was obtained. Thus, it was confirmed that the reproducibility was very excellent. The same FAD-dependent glucose dehydrogenase was immobilized on a carbon electrode, electric current measurement was conducted by amperometry at various glucose concentrations, and trial calibration was conducted. However, highly reliable results could not be obtained in this case due to the poor reproducibility of the data.

### Example 3

An enzymatic reaction was conducted as described in Example 1 further adding as an additive one of triethanolamine, Tricine, imidazole and collidine at a final concentration of 7 mM.

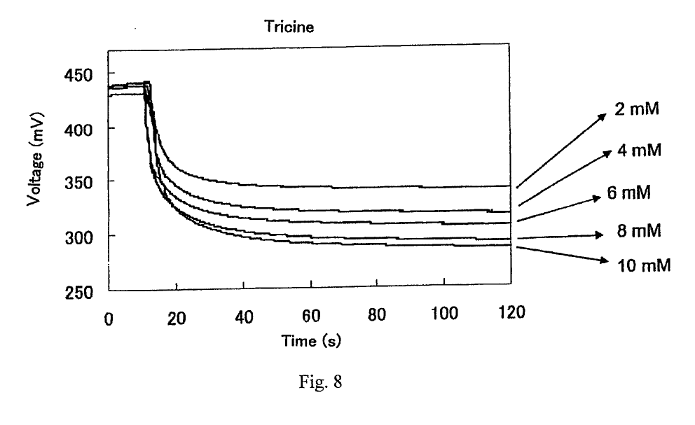

A working electrode and a reference electrode were set as described in Example 1 and connected to the general-purpose electrochemical measurement apparatus. Then, 2.4, 4.8, 7.2, 9.6 or 12 μl of a 1 M glucose solution was added thereto, and the electric potential value was immediately measured over time in each case. The glucose concentrations in the reaction systems were 2, 4, 6, 8 and 10 mM, respectively. Blank measurement was also conducted without the addition of glucose. As an example, the measurement results for the addition of Tricine are shown in FIG. 8. It was observed that as the enzymatic reaction proceeded, the electric potential declined over time and became steady after a while. It was confirmed that the increased amount of glucose added as a substrate resulted in the greater decline in electric potential. No change in electric potential was observed for the blank measurement without the addition of glucose.

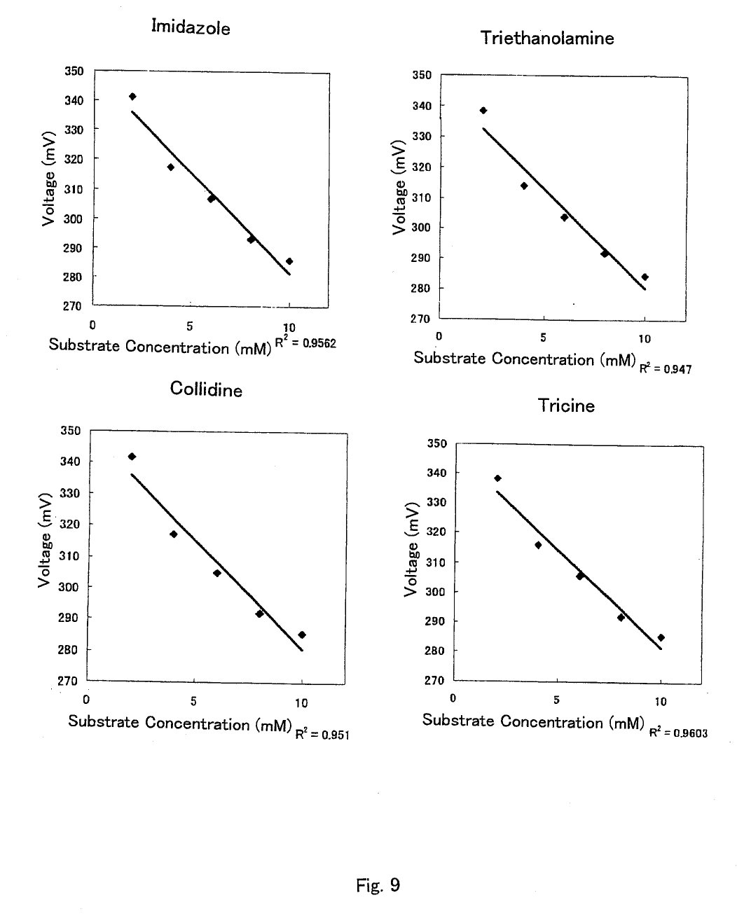

Measurements were carried out in the presence of the four kinds of additives, and calibration curves were prepared by plotting the electric potential values in the steady state against the glucose concentrations. The results are shown in FIG. 9. The electric potential value observed 120 seconds after the initiation of reaction was plotted along the longitudinal axis. Correlation coefficient values R² obtained by conducting regression calculation as shown in FIG. 9 were very high in all cases, and very good correlation was exhibited. It was suggested that the amount of glucose in the solution could be accurately quantified by measuring the electric potential in the steady state according to the method of the present invention.

### Example 4

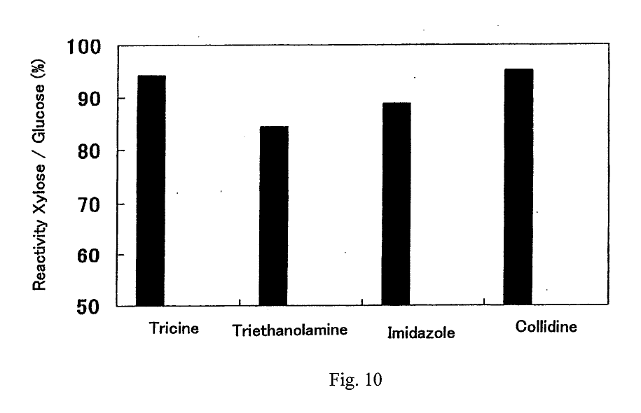

Furthermore, reaction selectivity for xylose was examined using various conditions. Measurements were conducted as described in Example 3 adding glucose or xylose at a final concentration of 5 mM. The ratios of decreased electric potentials observed upon measurements using xylose or glucose were calculated and are shown in FIG. 10. The effect of increasing the reaction selectivity for xylose was shown particularly when triethanolamine or imidazole was used.

### Example 5

200 μl of a 500 mM potassium ferricyanide solution (final concentration of 100 mM) and 10 μl of 10.6 kU/ml FAD-dependent glucose dehydrogenase (corresponding to 106 U) were added to a 2-ml water-jacketed glass cell (BAS) 650 μl of a 150 mM phosphate buffer (pH 7.0) was further added thereto to result in a total volume of 1 ml including the volume of a 1 M glucose solution to be added later. The mixture was slowly stirred using a stirrer. One having the mutation of G163R+V551C in SEQ ID NO:2 was used as the FAD-dependent glucose dehydrogenase. Furthermore, 100 μl of a 100 mM solution of iodoacetic acid (MIA), iodoacetamide (IAA) or sodium fluoride (NaF) was added as an additive (final concentration of 0.1 mM). The temperature was made constant at 30° C. during the enzymatic reaction by circulating water maintained at 30° C. in a thermostat bath through the water jacket part.

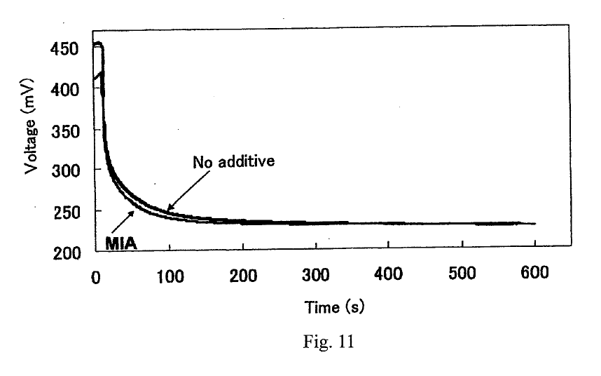

A working electrode and a reference electrode were set as described in Example 1 and connected to the general-purpose electrochemical measurement apparatus. Then, 40 μl of a 1 M glucose solution was added thereto, and the electric potential value was immediately measured over time in each case. The glucose concentration in the reaction system was 40 mM. As an example, the measurement results with the addition of iodoacetic acid (MIA) and without the addition of an additive are shown in FIG. 11. It was observed that as the enzymatic reaction proceeded, the electric potential declined over time and became steady after a while. It was confirmed that the addition of MIA resulted in the greater electric potential declining velocity. No change in electric potential was observed for blank measurement without the addition of glucose.

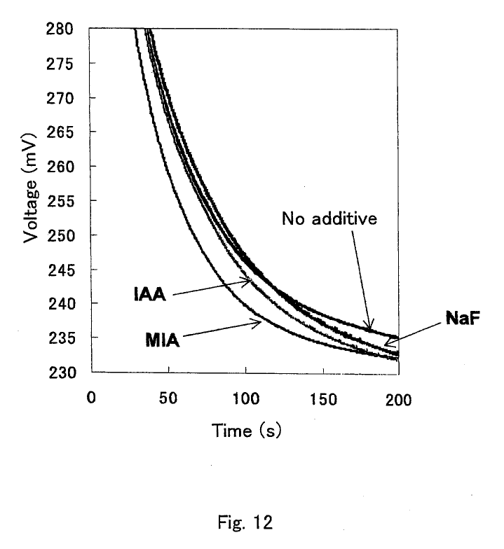

The measurement results with the addition of the three halogen compounds are shown in FIG. 12. The electric potential declining velocity was greater in the systems with the addition of IAA, MIA or NaF as compared with the case of no addition. Thus, the coexistence with a halogen compound could increase the initial velocity of the enzymatic reaction, resulting in shortened glucose measurement time. It was suggested that the amount of glucose in the solution could be accurately quantified by measuring the electric potential in the steady state according to the method of the present invention.

Regarding Examples 1, 2, 3 and 5, equivalent results were obtained when measurements were conducted according to the method as described in the respective Examples using glucose dehydrogenases each having a mutation of any one of the following in SEQ ID NO:2: K120E, G160E, G160I, G160P, G160S, G160Q, S162A, S162C, S162D, S162E, S162F, S162H, S162L, S162P, G163D, G163K, G163L, G163R, S164F, S164T, S164Y, L165A, L165I, L165N, L165P, L165V, A166C, A166I, A166K, A166L, A166M, A166P, A166S, S167A, S167P, S167R, S167V, N169K, N169P, N169Y, N169W, L170C, L170F, S171I, S171K, S171M, S171Q, S171V, V172A, V172C, V172E, V172I, V172M, V172S, V172W, V172Y, A180G, V329Q, A331C, A331D, A331I, A331K, A331L, A331M, A331V, K369R, K471R, V551A, V551C, V551T, V551Q, V551S, V551Y, (G160E+S167P), (G160I+S167P), (G160S+S167P), (G160Q+S167P), (S162A+S167P), (S162C+S167P), (S162D+S167P), (S162D+S167P), (S162E+S167P), (S162F+S167P), (S162H+S167P), (S162L+S167P), (G163D+S167P), (S164F+S167P), (S164T+S167P), (S164Y+S167P), (L165A+S167P), (L165I+S167P), (L165P+S171K), (L165P+V551C), (L165V+V551C), (A166C+S167P), (A166I+S167P), (A166K+S167P), (A166K+S167P), (A166M+S167P), (A166P+S167P), (A166S+S167P), (S167P+N169K), (S167P+N169P), (S167P+N169Y), (S167P+N169W), (S167P+L170C), (S167P+L170F), (S167P+S171I), (S167P+S171K), (S167P+S171M), (S167P+S171Q), (S167P+S171V), (S167P+V172A), (S167P+V172C), (S167P+V172E), (S167P+V172I), (S167P+V172M), (S167P+V172S), (S167P+V172T), (S167P+V172W), (S167P+V172Y), (S167P+V329Q), (S167P+A331C), (S167P+A331D), (S167P+A331I), (S167P+A331K), (S167P+A331L), (S167P+A331M), (S167P+A331V), (G163K+V551C).

### Example 6

An enzymatic reaction was conducted as described in Example 1 using as an FAD-dependent glucose dehydrogenase one consisting of the amino acid sequence of SEQ ID NO:3 in place of the one consisting of the amino acid sequence of SEQ ID NO:2.

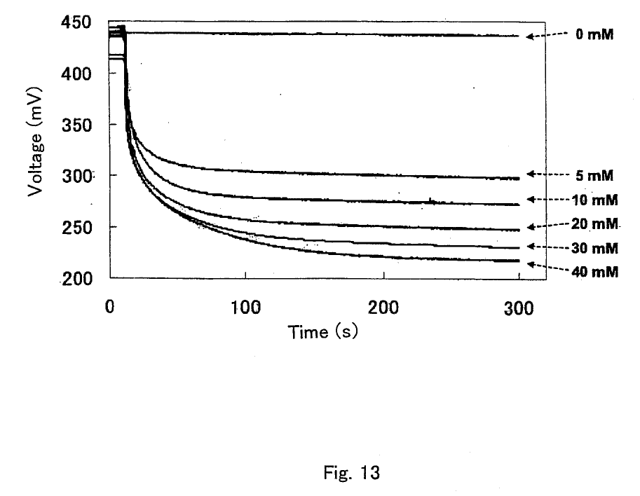

A working electrode and a reference electrode were set as described in Example 1 and connected to the general-purpose electrochemical measurement apparatus. Then, 6, 12, 24, 36 or 48 μl of a 1 M glucose solution was added thereto, and the electric potential value was immediately measured over time in each case. The glucose concentrations in the reaction systems were 5, 10, 20, 30 and 40 mM, respectively. Blank measurement was also conducted without the addition of glucose. The measurement results are shown in FIG. 13. It was observed that as the enzymatic reaction proceeded, the electric potential declined over time and became steady after a while. It was confirmed that the increased amount of glucose added as a substrate resulted in the greater decline in electric potential.

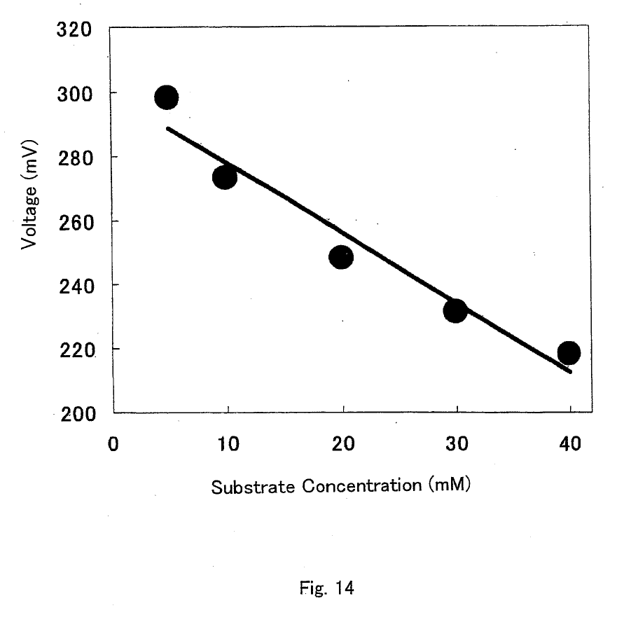

A calibration curve was prepared by plotting the electric potential values in the steady state in FIG. 13 against the glucose concentrations. The results are shown in FIG. 14. The electric potential value observed 300 seconds after the initiation of reaction was plotted along the longitudinal axis. A correlation coefficient R²=0.9478 was obtained upon regression calculation, and very good correlation was exhibited within a wide concentration range up to 40 mM. It was suggested that the amount of glucose in the solution could be accurately quantified by measuring the electric potential in the steady state according to the method of the present invention.

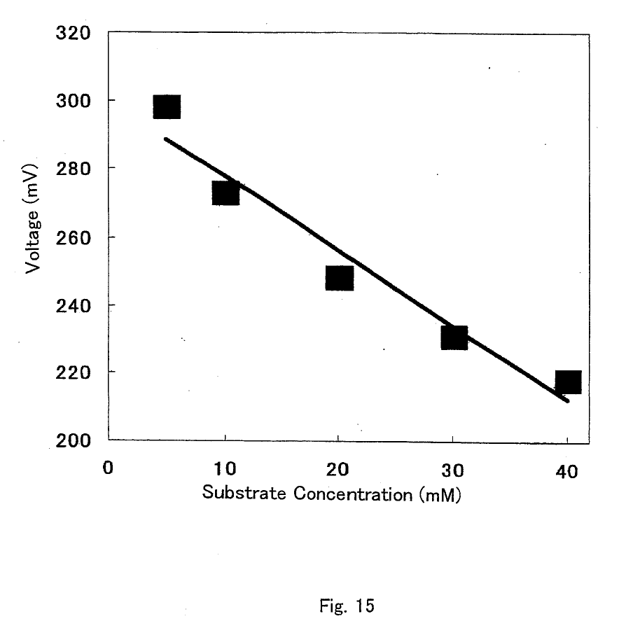

Furthermore, the reproducibility of the above-mentioned measurement was examined. The above-mentioned measurement was conducted once more. The results are shown in FIG. 15. As a result, a calibration curve almost identical to that in FIG. 14 was obtained. Thus, it was confirmed that the reproducibility was very excellent. The same FAD-dependent glucose dehydrogenase was immobilized on a carbon electrode, electric current measurement was conducted by amperometry at various glucose concentrations, and trial calibration was conducted. However, highly reliable results could not be obtained in this case due to the poor reproducibility of the data.

### Example 7

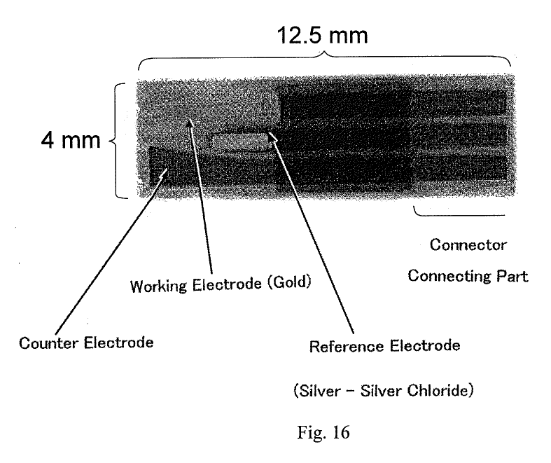

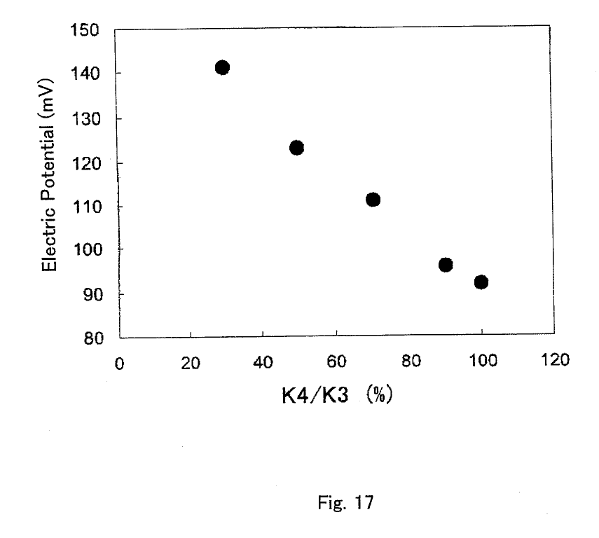

Solutions of total volumes of 60 μl with various compositions were prepared by changing the mixing ratios between a 10 mM potassium ferrocyanide solution and a 10 mM potassium ferricyanide solution (both in PBS). A DEP Chip electrode (gold, square; Bio Device Technology) as shown in FIG. 16 was immersed in one of the respective solutions. The electrode was connected to a general-purpose electrochemical measurement apparatus potentio/galvanostat model 1112 (Fuso Seisakusho) using a special connector for DEP Chip, and the electric potential was measured. The results of plotting of the composition ratio versus the electric potential are shown in FIG. 17. K4/K3 on the horizontal axis represents the percentage of the potassium ferrocyanide solution/the potassium ferricyanide solution.

As shown in FIG. 17, a high correlation was observed between K4/K3 and the electric potential. The action of the FAD-dependent glucose dehydrogenase on glucose results in the electron flow as shown in FIG. 1 and K3/K4 varies depending on the reaction level. Thus, quantitativeness in a wide range of glucose reaction level is suggested.

### Example 8

Glucose sensing by potentiometry was conducted using a DEP Chip electrode (gold, square; Bio Device Technology) as shown in FIG. 16. As to the composition of the reaction solution, a mixed solution of the following was prepared: 50 μl of PBS, 2 μl of 500 mM potassium ferricyanide and 5 μl of FAD-dependent glucose dehydrogenase (27.6 kU/ml; hereinafter also referred to as FAD-GLD). The electrode portion was immersed in the FAD-GLD solution, and connected to a general-purpose electrochemical measurement apparatus potentio/galvanostat model 1112 (Fuso Seisakusho) using a special connector for DEP Chip. One having the mutation of G163R+V551C in SEQ ID NO:2 was used as the FAD-dependent glucose dehydrogenase.

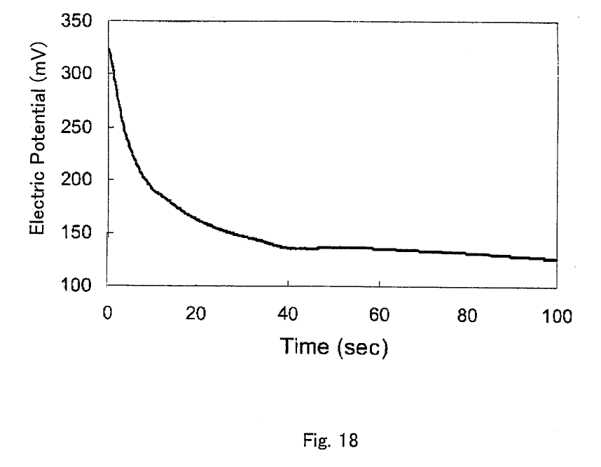

10 μl of a 100 mM glucose solution was added to the above-mentioned solution, and the change in the electric potential was measured. The results are shown in FIG. 18. It was observed that the electric potential became steady in about 40 seconds.

### Example 9

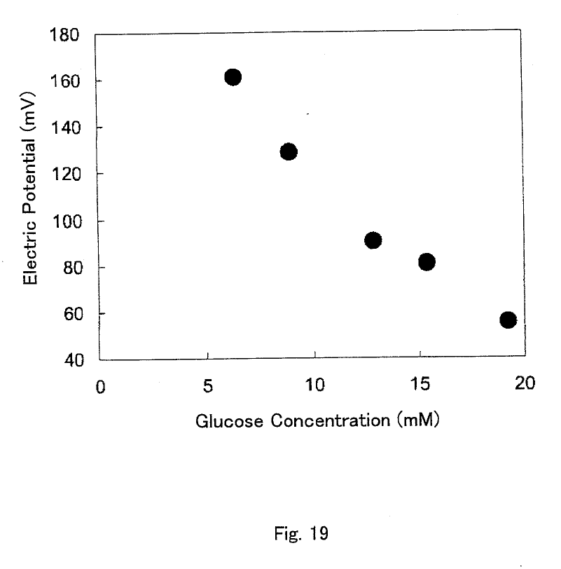

Potentiometry was conducted as described in Example 8 while changing the ratios between the glucose solution and PBS. The results of plotting of the relationship between the glucose concentration and the electric potential in the steady state are shown in FIG. 19. As shown in FIG. 19, excellent correlation was observed. It was confirmed that glucose can be quantified using this calibration curve.

### Example 10

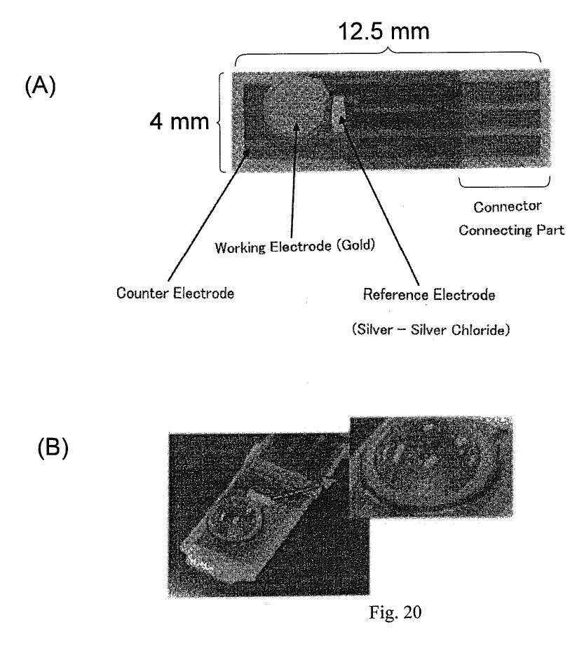

A DEP Chip electrode (gold, round-shaped; Bio Device Technology) as shown in FIG. 20(A) was mounted with 2 μL of a solution of 1 mM 7-carboxy-1-heptanethiol (hereinafter also referred to as MHA; Dojindo; see formula (I)) (ethanol:water=1:99 (volume ratio)) (see FIG. 20(B)). A carboxyl group surface was formed by standing in a humid environment at room temperature for 2 hours. MHA is an alkanethiol of 7 carbons having both a carboxyl group and a succinimide group at the ends of an alkyl chain.

The electrode was adequately washed with water and ethanol, and dried with air blow. The electrode was then mounted with 2 μL of a solution of 50 mM N-hydroxysulfosuccinimide (NHS)/200 mM 1-ethyl-3-[3-(dimethylamino)propyl]carbodiimide hydrochloride (EDC) (in PBS), and activated by standing in a humid environment at room temperature for 2 hours. Furthermore, the electrode was adequately washed with water and ethanol, and dried with air blow. The electrode was then mounted with 2 μL of a solution of 25 kU/ml FAD-dependent glucose dehydrogenase (hereinafter also referred to as FAD-GLD) (in PBS), and subjected to an immobilization reaction by standing in a humid environment at room temperature for 3 hours. One having the mutation of G163R+V551C in SEQ ID NO:2 was used as the FAD-dependent glucose dehydrogenase. The immobilization can be accomplished by coupling between the carboxyl group introduced onto the gold surface and the amino group of the FAD-GLD protein.

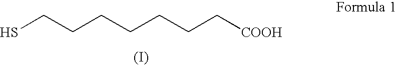

The electrode was adequately washed with water and dried with air blow. The electrode was then mounted with 2 μL of a solution of 1 mg/ml bovine serum albumin (BSA) (in PBS), and subjected to blocking of unreacted carboxyl groups by standing in a humid environment at room temperature for 1 hour.

The electrode prepared as described above was placed in a solution prepared by adding 5 μl of a 500 mM potassium ferricyanide solution to 80 μl of PBS, and connected to a general-purpose electrochemical measurement apparatus potentio/galvanostat model 1112 (Fuso Seisakusho) using a special connector for DEP Chip. 5 μl of a 100 mM glucose aqueous solution was added to the potassium ferricyanide solution, and the generated electric current was measured. The concept of this measurement system is illustrated in FIG. 1. The electric potential of potentio/galvanostat was set at 350 mV upon measurement.

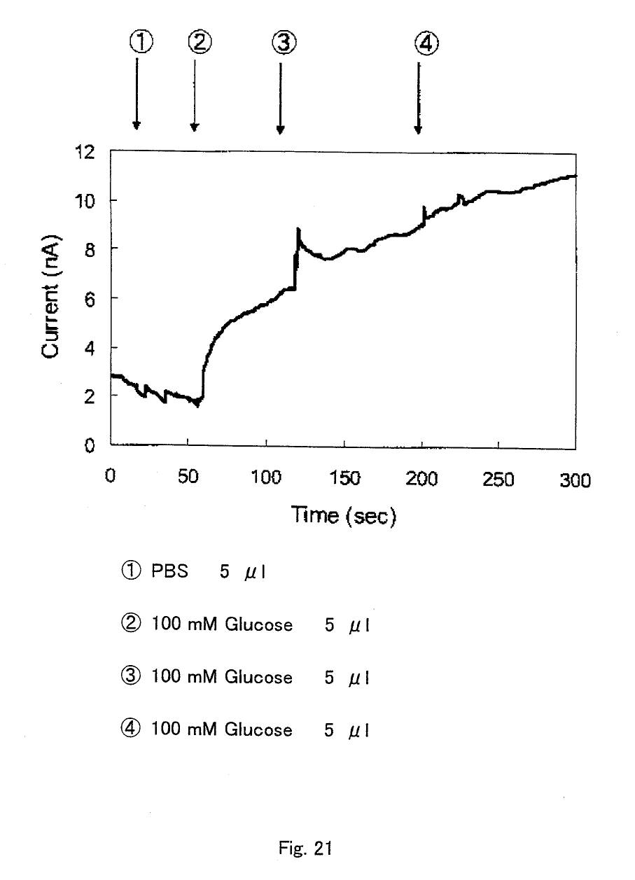

The measurement results by amperometry are shown in FIG. 21. It was observed that the electric current value was almost unchanged even when PBS was added, while addition of glucose elevated the electric current value.

### Example 11

A DEP Chip electrode (gold, round-shaped; Bio Device Technology) as shown in FIG. 20(A) was mounted with 2 μL of a solution of 2 mM PEG6-COONHS alkanethiol (SensoPath; SPT-0012C; see formula (II)) (ethanol:water=1:99 (volume ratio)) (see FIG. 20(B)). A succinimide group surface was formed by standing in a humid environment at room temperature for 2 hours. PEG6-COONHS alkanethiol is a PEG derivative having both a thiol group and a succinimide group at the ends of PEG. The electrode was adequately washed with water and ethanol, and dried with air blow. The electrode was then mounted with 2 μL of the solution of FAD-dependent glucose dehydrogenase (in PBS) as described in Example 10, and subjected to an immobilization reaction by standing in a humid environment at room temperature for 3 hours. The immobilization can be accomplished by reaction between the succinimide group introduced onto the gold surface and the amino group of the FAD-dependent glucose dehydrogenase protein.

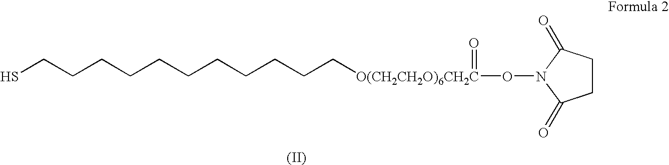

The electrode was adequately washed with water and dried with air blow. The electrode was then mounted with 2 μL of a solution of 2 mg/ml SUNBRIGHT (registered trademark) MEPA-20H (NOF Corporation) (in PBS), and subjected to blocking of unreacted succinimide groups by standing in a humid environment at room temperature for 1 hour. MEPA-20H is a PEG derivative having an amino group at the end, and its molecular weight is 2,000.

The electrode prepared as described above was placed in a solution of potassium ferricyanide in PBS under the conditions as described in Example 10, and connected to a general-purpose electrochemical measurement apparatus potentio/galvanostat model 1112 (Fuso Seisakusho) using a special connector for DEP Chip. 5 μl of a 100 mM glucose aqueous solution was added to the potassium ferricyanide solution, and the generated electric current was measured.

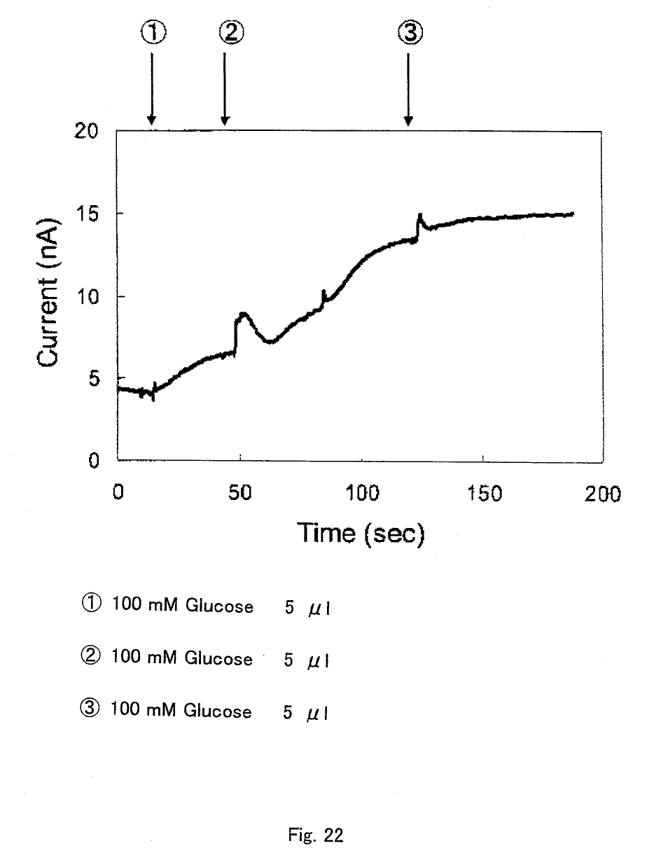

The measurement results by amperometry are shown in FIG. 22. Also in this case, it was observed that addition of glucose elevated the electric current value. Thus, excellent response to glucose could be observed by immobilizing a glucose dehydrogenase on a gold electrode.

Quantification of glucose in a solution can be realized conveniently with excellent reproducibility by utilizing the present invention. In particular, it is a technique useful for realizing rapid quantification, which is required in recent years, due to the effect of increasing the initial velocity of the enzymatic reaction. Thus, it is expected that its application to a blood glucose level sensor in the medical practice or to quality control of a glucose amount in the field of foods or the like is developed.

All publications and patent documents cited herein are hereby incorporated by reference in their entity for all purposes to the same extent as if each were so individually denoted.

What is claimed is:

1. A method of quantifying glucose in a solution, comprising measuring an electric potential by potentiometry using a glucose dehydrogenase that requires a flavin compound as a coenzyme.

2. The method of quantifying glucose according to claim 1, wherein the glucose dehydrogenase is a protein of (a) or (b) below:
(a) a protein consisting of the amino acid sequence of SEQ ID NO:1;
(b) a protein having a glucose dehydrogenase activity and consisting of an amino acid sequence in which one or several amino acid(s) is(are) delete, substituted or added in the amino acid sequence of SEQ ID NO:1.

3. The method of quantifying glucose according to claim 1, wherein the glucose dehydrogenase is a protein of (c) or (d) below:
(c) a protein consisting of the amino acid sequence of SEQ ID NO:2;
(d) a protein having a glucose dehydrogenase activity and consisting of an amino acid sequence in which one or several amino acid(s) is(are) delete, substituted or added in the amino acid sequence of SEQ ID NO:2.

4. The method of quantifying glucose according to claim 1, wherein the glucose dehydrogenase has an amino acid substitution at at least one position in SEQ ID NO:2 selected from the group consisting of position 120, position 160, position 162, position 163, position 164, position 165, position 166, position 167, position 169, position 170, position 171, position 172, position 180, position 329, position 331, position 369, position 471 and position 551.

5. The method of quantifying glucose according to claim 4, wherein the glucose dehydrogenase has at least an amino acid substitution of any one of the following in SEQ ID NO:2: K120E, G160E, G160I, G160P, G160S, G160Q, S162A, S162C, S162D, S162E, S162F, S162H, S162L, S162P, G163D, G163K, G163L, G163R, S164F, S164T, S164Y, L165A, L165I, L165N, L165P, L165V, A166C, A166I, A166K, A166L, A166M, A166P, A166S, S167A, S167P, S167R, S167V, N169K, N169P, N169Y, N169W, L170C, L170F, S171I, S171K, S171M, S171Q, S171V, V172A, V172C, V172E, V172I, V172M, V172S, V172W, V172Y, A180G, V329Q, A331C, A331D, A331I, A331K, A331L, A331M, A331V, K369R, K471R, V551A, V551C, V551T, V551Q, V551S, V551Y, (G160E+S167P), (G160I+S167P), (G160S+S167P), (G160Q+S167P), (S162A+S167P), (S162C+S167P), (S162D+S167P), (S162D+S167P), (S162E+S167P), (S162F+S167P), (S162H+S167P), (S162L+S167P), (G163D+S167P), (S164F+S167P), (S164T+S167P), (S164Y+S167P), (L165A+S167P), (L165I+S167P), (L165P+S171K), (L165P+V551C), (L165V+V551C), (A166C+S167P), (A166I+S167P), (A166K+S167P), (A166K+S167P), (A166M+S167P) (A166P+S167P), (A166S+S167P), (S167P+N169K), (S167P+N169P), (S167P+N169Y), (S167P+N169W), (S167P+L170C), (S167P+L170F), (S167P+S171I), (S167P+S171K), (S167P+S171M), (S167P+S171Q), (S167P+S171V), (S167P+V172A), (S167P+V172C), (S167P+V172E), (S167P+V172I), (S167P+V172M), (S167P+V172S), (S167P+V172T), (S167P+V172W), (S167P+V172Y), (S167P+V329Q), (S167P+A331C), (S167P+A331D), (S167P+A331I), (S167P+A331K), (S167P+A331L), (S167P+A331M), (S167P+A331V), (G163K+V551C), (G163R+V551C).

6. The method of quantifying glucose according to claim 1, wherein the glucose dehydrogenase has an amino acid substitution at at least one position in SEQ ID NO:2 selected from the group consisting of position 163, position 167 and position 551.

7. The method of quantifying glucose according to claim 6, wherein the glucose dehydrogenase has at least an amino acid substitution of any one of the following in SEQ ID NO:2: S167P, V551C, (G163K+V551C) and (G163R+V551C).

8. The method of quantifying glucose according to claim 1, wherein the glucose dehydrogenase exhibits an activity remaining ratio of 20% or more after heating at 50° C. for 15 minutes.

9. The method of quantifying glucose according to claim 1, wherein the glucose dehydrogenase exhibits a remaining activity of 80% or more after treatment at pH 4.5 to pH 6.5 at 25° C. for 16 hours.

10. The method of quantifying glucose according to claim 1, wherein the glucose dehydrogenase is derived from a filamentous fungus.

11. The method of quantifying glucose according to claim 10, wherein filamentous fungus belongs to the genus Penicillium or the genus Aspergillus.

12. The method of quantifying glucose according to claim 11, wherein the filamentous fungus belongs to Aspergillus oryzae.

13. The method of quantifying glucose according to claim 1, wherein a glucose reaction is detected by measuring a liquid junction potential in a solution of the glucose dehydrogenase that requires a flavin compound as a coenzyme, using a printed electrode in which a metal electrode is formed on an insulated substrate.

14. The method of quantifying glucose according to claim 13, wherein the detection of the glucose reaction is mediated by an electron transfer by a mediator.

15. An enzymatic reaction composition for measuring an electric potential by potentiometry, wherein a glucose dehydrogenase that requires a flavin compound as a coenzyme contained in the composition complies with one or more of the following:
(1) being dissolved in a Good's buffer
(2) coexisting with at least one compound selected from the group consisting of triethanolamine, Tricine, imidazole and collidine; and
(3) coexisting with a halogen compound.

16. The enzymatic reaction composition according to claim 15, wherein the Good's buffer is one or more selected from the group consisting of MOPS, PIPES, HEPES, MES, TES, BES, ADA, POPSO, Bis-Tris, Bicine, Tricine, TAPS, CAPS, EPPS, CAPSO, CHES, MOPSO, DIPSO, TAPS, TAPSO and HEPPSO.

17. The enzymatic reaction composition according to claim 15, wherein the glucose dehydrogenase coexists with as the halogen compound at least one compound selected from the group consisting of iodoacetic acid, iodoacetamide and sodium fluoride.

18. The enzymatic reaction composition according to claim 15, wherein the glucose dehydrogenase is a protein of (a) or (b) below:
(a) a protein consisting of the amino acid sequence of SEQ ID NO:1;
(b) a protein having a glucose dehydrogenase activity and consisting of an amino acid sequence in which one or several amino acid(s) is(are) delete, substituted or added in the amino acid sequence of SEQ ID NO:1.

19. The enzymatic reaction composition according to claim 15, wherein the glucose dehydrogenase is a protein of (c) or (d) below:
(c) a protein consisting of the amino acid sequence of SEQ ID NO:2;
(d) a protein having a glucose dehydrogenase activity and consisting of an amino acid sequence in which one or several amino acid(s) is(are) delete, substituted or added in the amino acid sequence of SEQ ID NO:2.

20. An electrochemical sensor for glucose measurement, in which a glucose dehydrogenase is covalently immobilized on a metal electrode via an alkanethiol or a hydrophilic macromolecule, and with which a glucose reaction is detected electrochemically.

21. The electrochemical sensor for glucose measurement according to claim 20, wherein the metal electrode is formed on an insulated substrate.

22. The electrochemical sensor for glucose measurement according to claim 20, wherein the metal electrode is round-shaped.

23. The electrochemical sensor for glucose measurement according to claim 22, wherein the radius of the metal electrode is 2 mm or less.

24. The electrochemical sensor for glucose measurement according to claim 20, wherein the hydrophilic macromolecule is polyethylene glycol (PEG).

25. The electrochemical sensor for glucose measurement according to claim 20, wherein a change in an electric current generated due to the action with glucose is measured.

26. The method of quantifying glucose according to claim 1, wherein the activity of the glucose dehydrogenase is inhibited in the presence of 1 mM 1,10-phenanthroline by 30% or more.

27. The method of quantifying glucose according to claim 26, wherein the glucose dehydrogenase is a protein of (a) or (b) below:
(a) a protein consisting of the amino acid sequence of SEQ ID NO:3;
(b) a protein having a glucose dehydrogenase activity and consisting of an amino acid sequence in which one or several amino acid(s) is(are) delete, substituted or added in the amino acid sequence of SEQ ID NO:3.

28. The method of quantifying glucose according to claim 26, wherein the glucose dehydrogenase is derived from the genus Penicillium or the genus Aspergillus.

29. The method of quantifying glucose according to claim 26, wherein the glucose dehydrogenase is derived from Aspergillus terreus.


SEQUENCE LISTING (3 sequences)

SEQ ID NO: 1 - protein, 593 aa, Aspergillus oryzae
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 2 - protein, 572 aa, Aspergillus oryzae
MKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 3 - protein, 568 aa, Aspergillus terreus
MKYDYIVIGGGTSGLAVANRLSEDPSVNVLILEAGGSVWNNPNVTNVNGYGLAFGSDIDWQYQSVNQPYGGNVSQVLRAGKALGGTSTINGMAYTRAEDVQIDAWETIGNTGWTWKNLFPYYRKSENFTVPTKSQTSLGASYEAGAHGHEGPLDVAFTQIESNNLTTYLNRTFQGMGLPWTEDVNGGKMRGFNLYPSTVNLEEYVREDAARAYYWPYKSRPNLHVLLNTFANRIVWDGEARDGDITASGVEITSRNGTVRVINAEKEVIVSAGALKSPAILELSGIGNPSVLDKYNIPVKVNLPTVGENLQDQVNSHMDASGNTSISGTKAVSYPDVYDVFGDEAESVAKQIRASLKQYAADTAQANGNIMKAADLERLFEVQYDLIFKGRVPIAEVLNYPGSATSVFAEFWALLPFARGSVHIGSSNPVEFPVINPNYFMLDWDAKSYVAVAKYIRRSFESYPLSSIVKESTPGYDVIPRNASEQSWKEWVFDKNYRSNFHPVGTAAMMPREIGGVVDERLNVYGTTNVRVVDASVLPFQVCGHLVSTLYAVAERAADLIKADAGRR